(19) United States
(12) Patent Application Publication
(10) Pub. No.: US 2024/0181186 A1
(43) Pub. Date: Jun. 6, 2024

(54) FOOT PEDAL UNIT FOR AIRWAY MASK

(71) Applicant: FUNDACION ABOOD SHAIO EN REESTRUCTURACION, Bogota (CO)

(72) Inventors: Maria Lucia Arango Cortes, Bogota (CO); Carlos Javier Latorre Rojas, Bogota (CO); Jose Luis Lozano Villanueva, Bogota (CO); Nicolas Navarro Pulido, Bogota (CO)

(21) Appl. No.: 17/759,645

(22) PCT Filed: Apr. 23, 2021
(86) PCT No.: PCT/IB2021/053354

§ 371 Date: Jul. 28, 2022

(51) Int. Cl. A61M 16/00; A61M 16/06; A61M 16/08; A61M 16/20; F16K 11/00

(52) U.S. Cl. CPC A61M 16/0075; A61M 16/0616; A61M 16/0816; A61M 16/201; F16K 11/00; A61M 2016/0027; A61M 2205/078

(57) ABSTRACT

The invention belongs to the field of medical sciences and respiratory masks. It is a cardiopulmonary resuscitation device that delivers assisted ventilation via a foot- or compressor-controlled bellows mechanism. This device frees the use of the hand used to compress the conventional self-inflating Airway Mask Bag Unit (AMBU), so that the free hand can be used to fit the mask over the patient's face and reduce the mechanical fatigue generated by the manual compression of current mechanisms. The unit attaches to ventilation masks, can have a frequency meter and is compatible with accessories comprising conventional cardiopulmonary resuscitation devices; inside it has a valve that controls the filling of the oxygen reservoir and the time at which oxygen is released to be supplied to the patient.

### TECHNOLOGY SECTOR

The present invention belongs to the field of medical or veterinary sciences; hygiene, respiratory or anesthesia masks, and more particularly to a device for cardiopulmonary resuscitation and has the purpose of providing assisted ventilation to the patient by means of a bellows mechanism controlled by the foot. This mechanism frees the use of the hand that is regularly used to compress the conventional self-inflating bag or also called AMBU (Airway Mask Bag Unit), in order to be used in the correct adjustment of the mask on the patient's face and ensure a proper seal, as well as reducing the mechanical fatigue generated by the manual compression that is performed with the current mechanisms. The unit is coupled to conventional ventilation masks and has a valve inside that controls the filling of the oxygen reservoir and the moment in which the oxygen is released to be supplied to the patient.

### STATE OF THE ART

Conventional ventilation is routinely performed in cases of cardiopulmonary pulmonary resuscitation and in most cases is done with a self-inflating bag system or also called AMBU (Airway Mask Bag Unit), which is connected to a face mask that allows the passage of air from the bag to the respiratory tract, However, it is important to describe that in order to achieve effective ventilations, it is necessary to make a good seal between the face mask and the patient's face, a seal that is achieved by positioning both hands around the mask leaving room for the traction of the jaw. Likewise, to achieve air entry into the mask, adequate insufflation of the balloon should be performed, pressing the balloon for each ventilation required. Current devices are not designed to optimally guarantee the performance of both functions simultaneously, since to ensure an adequate seal of the face mask it is necessary to perform a bimanual maneuver on the patient's face and the balloon, in order to guarantee the stability of the mask and avoid air loss. When balloon insufflation is required, the operator must necessarily remove one of his hands from the face mask in order to depress the balloon containing the mechanism and send the air into the respiratory tract, so that the proper seal between the face mask and the nasal and oral cavities of the patient is lost, originating in most cases, air leakage and loss of the effectiveness of the ventilations.

These failures are even more prevalent in cases where the health professional in charge of performing ventilations does not have adequate training, if simultaneous maneuvers are being carried out on the individual's airway or even if the patient has factors associated with difficult ventilation. Likewise, the physical effort involved in the proper positioning of the mask and the simultaneous performance of balloon insufflation for a prolonged period of time, as in cases of cardiopulmonary resuscitation, causes the operator to make a greater mechanical effort as the minutes go by, which increases the physical wear and tear and can generate even more errors in the process.

In terms of relevant background, there is patent application No. EP3020439A1 “Ventilation Device and method for operating a ventilation device”, published on May 18 2016 and whose holder is Vera Seifert, which shows a respiratory device comprising a respiratory bag having at least one oxygen-containing gas inlet valve, an oxygen-containing gas outlet valve, a respiratory element and a mechanism actuation device comprising a foot pedal. The pedal can be configured as a lever, button, switch, rocker, press, pliers or any combination of these. It is also possible to design the foot control device and the resuscitation bag as a bellows. When operating all of the above-mentioned embodiments, a rectilinear or arcuate (lifting) motion is performed with the foot. The present invention refers to a respiratory device and method oriented to facilitate the handling of manual resuscitators and AMBU respiratory bags, mainly seeks freeing at least one hand when providing therapy by means of an actuator element designed as a pedal of multiple configurations that depresses the respiratory bag, which has oxygen inlet and outlet valves. One of the main differences lies in the fact that the prior art does not describe the O2 reservoir bag but mentions that O2 is supplied directly by means of O2 inlet and outlet valves.

The claims of the invention EP302020439A1 comprise a complete system of device for manual resuscitation. On the other hand, the present invention describes a unique accessory that replaces the ambu-bag, comprising a pedal-operated flexible bellows mechanism that initially supplies air but which, thanks to being compatible with the masks and different valves existing on the market, it is possible to connect it with the pressure valves and thus with the O2 reservoir bags. In addition, it has a valve system that includes a gate to ensure the correct filling of the O2 reservoir bag.

There is also the Patent Application No. US20080087285A “Adjustable Multifunctional Carrying Strap for an AMBU-bag,” published on Apr. 17 2008, which shows an adjustable multifunctional carrying strap for an AMBU-bag attached to the ambu-bag and can be adjusted to be tighter or looser. The carrying strap allows a single palm to be attached to the ambu-bag, so that the ambu-bag can be operated with one hand, and a mask can be operated with the other hand to press on the jaw part of a patient, so as to effectively achieve a rescue purpose. In addition, the carrying strap may also allow the ambu-bag to be carried with one hand or hung on a wall as a backrest. In this background, the strap allows the ambu-bag to be held more firmly, however, the mechanical wear generated by the hand movement for compression of the bag remains, likewise, holding the patient's mask with one hand alone does not guarantee a proper seal of the mask. The new invention is part of devices for cardiopulmonary resuscitation and is intended to provide assisted ventilation to the patient by means of a bellows mechanism controlled by the foot; it provides a new mechanism for filling the air pedal in the unit by means of a damper which in turn controls the oxygen inlet into the mask from the reservoir bag and from the external supply.

The invention is intended to function as an accessory that replaces the manual resuscitation bag, but is compatible with other existing accessories such as mask, pressure valves, connection to oxygen supply, among others, and is also suitable for use with external oxygen supply or only with ambient air supply.

Additionally, patent application US20160067435A1, “Endopharyngeal Airway Device and Kit and Method of Use,” published on Mar. 10, 2016, shows A kit for ventilating a patient incapable of normal ventilation with current anesthetic techniques due to a risk for airway obstruction in surgical procedures requiring conscious sedation monitored anesthesia care (MAC). Includes a single-lumen endopharyngeal nasal tube with an eye shaped opening at its distal end and a cylindrical proximal end used as an adaptor for anesthetic applications such as positive airway pressure via modified ambu-bag or other various anesthesiology applications. Contiguous to the distal end of the nasal tube is an end-tidal CO2 monitoring port (ETCO2). On the distal end of the flexible nasal ventilation tube is an eye-opening allowing air flow to the posterior pharynx. The kit further includes a modified ambu-bag which allows for controlled airflow to the patient during a procedure.

This background discusses a modification of the anesthesia delivery system for patients requiring partial consciousness during surgical procedures, especially designed for patients with airway obstruction. The device is intended to improve the flow and direction of air over the patient, does not have automatic insufflation control systems and is used with positive pressure equipment. Additionally, the background requires a type of intubation which is not necessary in cardiopulmonary resuscitation and is focused on improving the anesthesia procedure with partial state of consciousness. Its design does not specify how to ensure the seal over the patient's face, nor does it reduce muscle fatigue due to compression of the insufflation bag, which are solutions proposed by the new invention.

### DESCRIPTION OF THE INVENTION

The attached figures illustrate the proposed scope of the invention within the proposed foot pedal insufflation unit for airway mask.

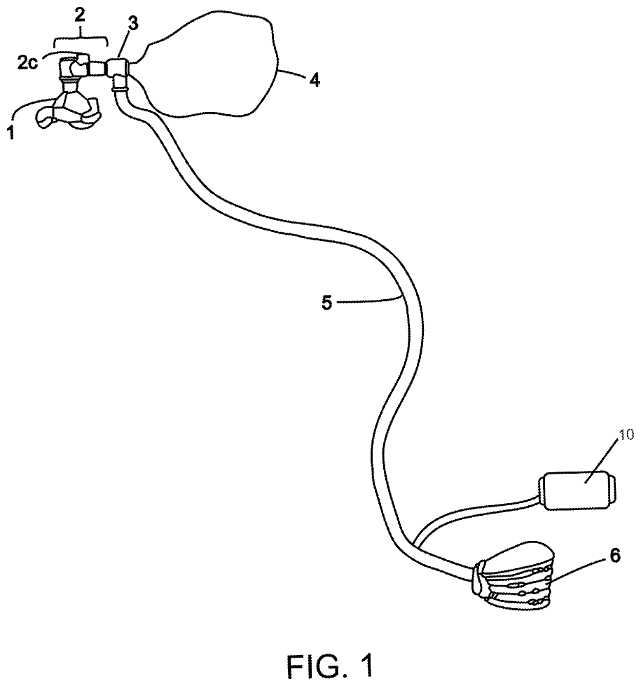

FIG. 1 shows a side view of the foot pedal insufflation unit for airway mask.

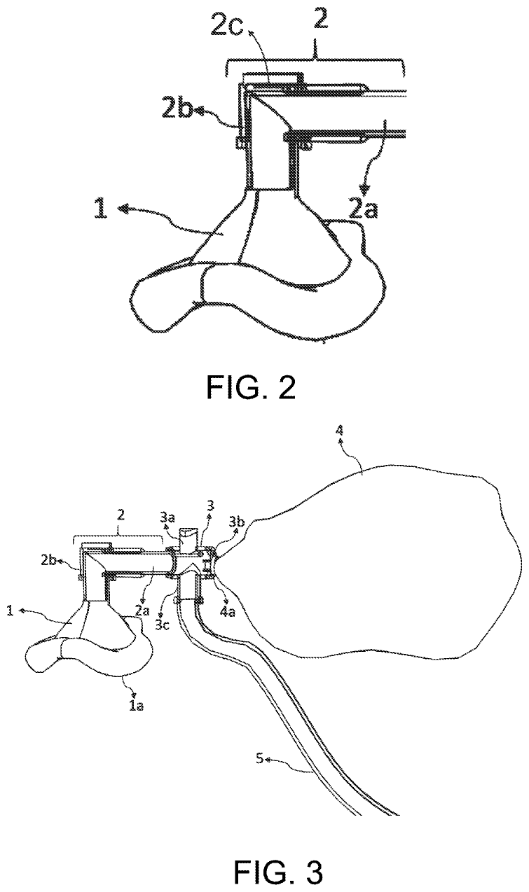

FIG. 2 shows an enlarged side view of the mask and its connection to the valve mechanism.

FIG. 3 shows a magnified side view of the mask and its relationship to the reservoir and the open connection of the foot-operated insufflator.

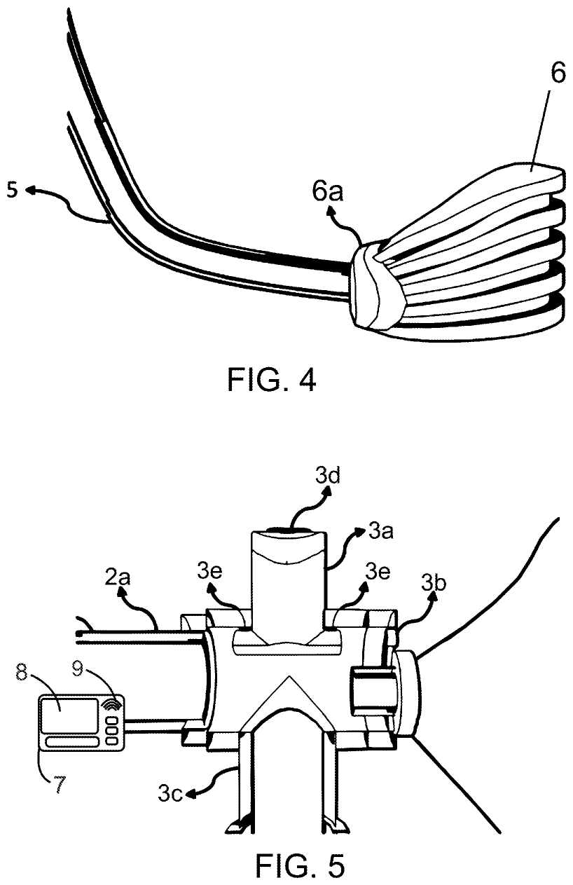

FIG. 4 shows a side view of the pedal insufflation unit.

FIG. 5 shows a side view of the part (3) and the closed state of the valve controlling the ambient air intake.

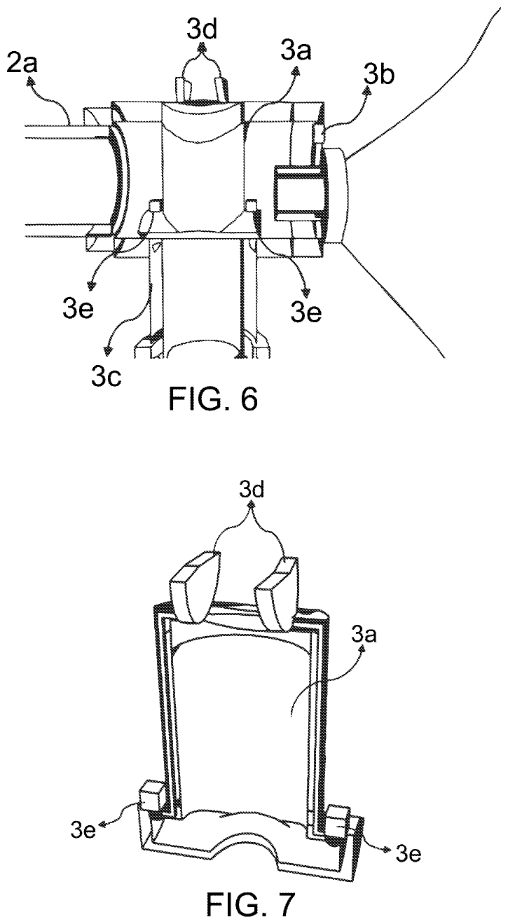

FIG. 6 shows a side view of part (3) and the open state of the valve controlling the ambient air intake.

FIG. 7 shows a graphical illustration of the hollow gate (3) and the relationship of the actuators to the valve (3d).

The present invention is a foot-operated insufflation unit for an airway mask which is part of cardiopulmonary resuscitation devices and has the purpose of providing assisted ventilation to the patient by means of a foot-controlled bellows mechanism. This mechanism frees the use of the hand that is regularly used to compress the conventional ambu-bag, in order to be used in the correct adjustment of the mask on the patient's face and ensure a proper seal, also reduces mechanical fatigue generated by the manual compression that is performed with the current mechanisms.

The unit is coupled to conventional ventilation masks and has five main parts inside: a valve that controls the filling of the oxygen reservoir and the moment in which oxygen is released to be supplied to the patient, an airtight mask, a reservoir, a pedal and a pedal connector tube.

The device comprises a mask (1) with perimetral hermetic seal (1a) that is placed over the patient's face that when pressed generates a hermetic seal between the face and the mask (1). A coupling (2) connects the mask (1) to the valve mechanism (3) through the connector (2a) which also has a pressure measuring device (2c) to indicate the pressure at which the air is being insufflated and a perpendicular connector (2b) that adjusts the pressure measuring device (2c) to the connector (2a).

The valve mechanism (3) is connected to the pedal tube (5) through the perpendicular section (3c), where the perpendicular section (3c) has a damper (3a) that controls the passage of air from the pedal tube (5) and a reservoir (4) when the pressure is sufficient to raise the damper (3a).

The valve mechanism (3) is connected to the reservoir (4) with a reservoir coupling (4a), in the reservoir oxygen is stored which flows from external administration and is connected to the damper (3b) which conducts oxygen to the reservoir (4) and the valve mechanism (3). A bellows pedal (6) is connected to the pedal tube (5), which when depressed, generates a stream of air flowing through the pedal tube (5) to the connector (3c) over which is located the damper (3a) which is raised allowing the mixing of the air generated by the bellows pedal (6) with oxygen from the reservoir (4) and with oxygen from the external source entering through the damper (3b); where this air mixture is directed to the mask (1) through the connector (2) and in this way is supplied to the patient through the mask (1) held tightly on his face.

Thus, by facilitating the use of both hands in the maneuver of securing the mask (1) on the patient's face, a better oxygen transmission is guaranteed and therefore the cardiopulmonary resuscitation process. On the contrary, when the bellows pedal (6) is not depressed, the damper (3a) remains static, which facilitates the filling of the reservoir (4) with oxygen from an external source that enters through the damper (3b).

FIG. 4 shows the bellows pedal (6) and the connection (6a) joining it to the pedal tube (5) that conducts air to the connector (3c) to be distributed to the rest of the device.

The damper (3a) is hollow inside and has a valve (3d) which controls the entry of ambient air through the connector (3c) to be carried through the pedal tube (5) and finally reach the bellows pedal (6). In this way, the bellows pedal (6) will have the necessary fluid to propel the oxygen from the reservoir back into the mask (1). The valve (3d) closes when the bellows pedal (6) is depressed, because the actuators (3e) which are connected to the valve (3d), are in turn depressed by the inner wall of the connector (3) as shown in FIG. 5, generating the closing of the valve (3d) by a mechanical, hydraulic, pneumatic or electronic closing mechanism as shown in FIG. 7. In FIG. 6 the actuators (3e) are shown released, this occurs when the bellows pedal (6) is not depressed, therefore, when the actuators (3e) are released the valve (3d) opens and allows ambient air to enter through the connector (3c) and then the pedal tube (5) to finally reach the bellows pedal (6). The valve (3d) has a conventional and commercially available impurity filter that prevents the entry of unwanted particles.

In one embodiment of the invention, the device has a frequency meter (7) with LED display (8) for visualization of the frequency signals and a signal transmitter (9) that transmits the data to an external display and allows estimating the frequency and ventilatory response of the patient to facilitate the decrease of respiratory acidosis without generating barotrauma, by measuring exhaled CO2 continuously; which in turn serves to measure the respiratory frequency and the measurement of oxygen saturation SPO2, which is also useful in the estimation of heart rate.

When the patient is on the floor and the bellows pedal (6) cannot be actuated due to the position of the healthcare personnel, a compressor (10) is actuated which accumulates air and subsequently releases this air towards the patient in a synchronous manner, i.e. the insufflation is automated based on the measurement of the different sensors.

Airway Mask Bag Unit (AMBU) ventilation must maintain adequate systemic oxygenation in order to maintain basal body requirements and hemodynamic stability. Therefore, the new invention of a foot-operated insufflation unit for airway mask provides a ventilation solution that allows the operator to acquire a favorable position to perform the mask seal (1) and balloon insufflation, in order to achieve effective ventilations, reducing the resistance to the entry and exit of fresh gases and reducing both the excessive loss of air in the system and the physical effort made by the health professional in charge, by improving the ergonomics to carry out the maneuver.

1. Pedal insufflation unit for airway mask CHARACTERIZED by a device with five main parts: A first part called mask (1) with a perimeter hermetic seal (1a) which is placed over the patient's face on one side and a coupling (2) connecting the mask (1) with a valve mechanism (3) through the connector (2a); a second part called valve mechanism (3) which has a pressure measuring device (2c) to indicate the pressure at which the air is being insufflated, has a perpendicular connector (2b) that adjusts the pressure measuring device (2c) to the connector (2a), a damper (3b) for external oxygen access, a frequency meter (7) and a reservoir coupling (4a) for connection to a reservoir (4); wherein the valve mechanism (3) is connected to the pedal tube (5) through the perpendicular section (3c) which has a gate (3a) that controls the passage of air coming from a pedal tube (5) and is connected to the reservoir (4); a third part is the reservoir (4) which runs when the pressure is sufficient to raise the damper (3a); a fourth part refers to the bellows pedal (6) or compressor (10) that provides the necessary fluid to propel the oxygen from the reservoir into the mask (1) connected to the fifth part called pedal tube (5) connected at its opposite end to the valve mechanism (3), so that when the bellows pedal (6) is depressed, generates a stream of air flowing through the pedal tube (5) to the connector (3c) on which is located the damper (3a) that rises allowing the mixing of the air generated by the bellows pedal (6) or the air generated by the compressor (10) with the oxygen from the reservoir (4) and with the oxygen from the external source entering through the damper (3b).

2. Pedal insufflation unit for airway mask according to claim 1, CHARACTERIZED in that the valve mechanism (3) is connected to the reservoir (4) with a reservoir coupling (4a), wherein the reservoir stores oxygen flowing from external administration and is connected to the damper (3b) which conducts oxygen to the reservoir (4) and the valve mechanism (3).

3. Pedal insufflation unit for airway mask according to claim 1, CHARACTERIZED in that the damper (3a) is hollow inside and has a valve (3d) which controls the entry of ambient air through the connector (3c) to be carried by the pedal tube (5) to finally reach the bellows pedal (6).

4. Pedal insufflation unit for airway mask according to claim 1, CHARACTERIZED in that the valve mechanism (3) has actuators (3e) with which are connected to the valve (3d) with mechanical, hydraulic, pneumatic or electronic type locking mechanism and conventional particulate filter, wherein the actuators (3e) are in turn depressed by the inner wall of the connector (3).

5. Pedal insufflation unit for airway mask according to claim 1, CHARACTERIZED in that the frequency meter (7) has LED display (8) for visualization of the frequency signals and a signal transmitter (9) that transmits the data to an external display, which estimates the frequency and ventilatory response of the patient by measuring exhaled CO2 continuously.
